(19) United States
(12) Patent Application Publication
(10) Pub. No.: US 2011/0139634 A1
(43) Pub. Date: Jun. 16, 2011

(54) SYSTEM AND METHOD FOR MEASURING ANALYTE CONCENTRATION WITH INTERFERANT CORRECTION

(75) Inventors: Tai-Cheng Chou, Shulin City (TW); Chia-chi Wu, Kaohsiung City (TW); Tsai-Yun Lee, Sindian City (TW); Chao-Wang Chen, Taipei City (TW)

(73) Assignee: Taidoc Technology Corporation

(21) Appl. No.: 12/960,777

(22) Filed: Dec. 6, 2010

(30) Foreign Application Priority Data
Dec. 14, 2009 (TW) 98142805

(51) Int. Cl. G01F 1/64; G01N 33/487

(52) U.S. Cl. 205/792; 204/403.02

(57) ABSTRACT

The present invention is related to an electrochemical biosensing test strip, biosensing meter, system and method for analyte measurement incorporating a hematocrit correction. The biosensor strip comprises a first and a second electrode sets respectively for detecting analyte concentration and hematocrit level. The first and second electrode sets are respectively corresponding to different reaction zones and a reaction reagent is only formed on the first electrode set corresponding reaction zone. Applying a first signal comprising a DC component and a second signal comprising an AC component that has a constant frequency respectively to the first and second electrode sets can respectively detect an uncorrected analyte concentration and hematocrit level. Therefore, the corrected analyte concentration is more accurate by incorporating the hematocrit correction.

### BACKGROUND OF THE INVENTION

1. Field of the Invention

The present invention relates in general to a system and method for measuring analyte concentration with interferant correction. More particularly, the present invention relates to a biosensing meter, biosensor strip, system and method for measuring analyte concentration with hematocrit correction by applying AC and DC signals simultaneously.

2. Description of the Related Art

Since the improvement of the science and technology, many tests can be operated by users at home. In the market, many disposable strips are used for measuring specific components in a biological fluid and can be operated by users at home. Analytical biosensor strips are useful in chemistry and medicine to determine the presence and concentration of a biological analyte. Such strips are needed, for example, to monitor glucose in diabetic patients and lactate during critical care events. In the recent year, Diabetes is a modern disease, especially in elders. Most people need an accurate measurement of blood glucose.

Conventional electrochemical biosensor strip has a base, an electrode system, an insulating substrate, a test reagent and a cover. The electrode system is laid on the base and comprises two electrodes separated from each other. The insulating substrate is laid down onto the electrode system and has a first opening and a second opening. The first opening exposes portions of the electrode system for electrical connection with a mating meter, which measures some electrical property of a test sample after the test sample is mixed with the test reagent of the strip. The second opening exposes a different portion of the electrode system for application of the test reagent to those exposed surfaces of electrode system. The test reagent is a reagent that is specific for the test to be performed by the strip. The test reagent may be applied to the entire exposed surface area of the electrode system in the area defined by the second opening. The cover is covered on the electrode system and the test reagent for protecting the test reagent. When the test sample received, it contacts with the test reagent and the electrode system transfers an electrical signal that correlates to the concentration of an analyte being measured in the test sample.

Electrochemical method is one of the typically method for measuring analyte concentration and involves amperometric responses indicative of the concentration of the analyte. An important limitation of electrochemical methods of measuring the concentration of the analyte in blood is the effect of confounding variables on the diffusion of analyte and the various active ingredients of the reagent. Electrochemical method has a problem that the accuracy of the test is interfering by hematocrit (a ratio of the volume of packed red blood cells to the total blood volume).

The normal hematocrit range for a typical human being is about 35% to 45%, though in extreme cases, the hematocrit may range from about 20% to about 70%. The mean hematocrit range for neonatal is about 53% to 69%. Variations in a volume of red blood cells within blood can cause variations in glucose readings measured by electrochemical test strips. Typically, a negative bias (i.e., lower calculated analyte concentration) is observed at high hematocrit, while a positive bias (i.e., higher calculated analyte concentration) is observed at low hematocrit. At high hematocrit, the red blood cells may impede the reaction of enzymes and electrochemical mediators, reduce the rate of chemistry dissolution since there less plasma volume to solvate the chemical reactants, and slow diffusion of the mediator and then cause a slower current result. Conversely, at low hematocrit, a higher measured current can result. In addition, the blood sample resistance is also hematocrit dependent, which can affect voltage and/or current measurements.

Besides, variation of hematocrit is extremely broad, and therefore, it needs to measure hematocrit by biosensing meter and biosensor strip. It is very important to design a biosensor strip and biosensing meter having functions of preventing hematocrit interfering. How to make a system and a method for removing hematocrit interfering of analyte measurement is needed by present related manufactory.

U.S. Pat. No. 7,407,811 ('811) described a system and a method for analyte measurement by using AC excitation to measure hematocrit for decreasing hematocrit interfering. Further, the method of '811 is measuring phase angle and admittance magnitude of the AC excitation and cooperated with a formula to detect hematocrit. '811 further described blood glucose measurement for correcting hematocrit by using above hematocrit measurement method, which applying DC and AC signals in only one electrode set and only one reaction zone of a bioseneor strip, whether applying AC or DC signal firstly. It is measuring phase angle and admittance magnitude of AC excitation to detect hematocrit and DC excitation to detect analyte concentration. Further, parameters of a set formula of the prior method further include temperature, and therefore, the analyte concentration will be corrected with the phase angle, admittance magnitude and temperature. Besides, the provided AC excitation used at least two frequencies and it used two to five frequencies in practice, and therefore, the hematocrit is detected by applied AC excitation with different frequencies.

However, the method of '811 is providing AC and DC signals to a sample in the same reaction zone and further used only one electrode set to detect, and therefore, there could be noise produced to interfere with each other. Besides, a result of uncorrected analyte concentration and hematocrit measured by provided AC with DC offset alone to the same reaction zone will interfere with each other result and then influence the accuracy. The method of '811 further needed temperature to correct the measured analyte concentration and needed over one AC frequency to go to the aim. It requires complicated operations and takes a long time. Furthermore, the cost and complexity of the meter increases as the number of measurements and frequencies increases. Thus, it is needed to provide a system and a method capable of solving the foregoing problem.

Besides, many sold electrochemical biosensor strips in the market have another problem in that a sample volume will also influence the accuracy. In measurement of blood glucose, for example, it is quietly sensitive to blood sample volume, and if the sample is insufficient that will cause an error of calculation. Thus, it is needed to solve the above advantage.

### SUMMARY OF THE INVENTION

In order to solve the above noted conventional problems, one aspect of the present invention is to provide a biosensor strip, biosensing meter, system and method that is measurement of hematocrit and analyte concentration at two electrode sets and two reaction zones respectively. Furthermore, one aspect of the present invention is to provide a biosensor strip, biosensing meter, system and method that the reaction zone for measurement of hematocrit has not covered with a reaction reagent.

An aspect of the present invention is provided a biosensor strip for measurement an analyte concentration, comprising:

a base;

an electrode layer covered on the base and comprising a first electrode set and a second electrode set, wherein the first electrode set is used for measurement analyte concentration and the second electrode set is used for measurement hematocrit level;

a space covered on the electrode layer and exposed an end of the electrode layer and comprising an opening, wherein the opening exposed another end of the electrode layer, and further comprises a separated element formed corresponding to and in the middle of the first electrode set and the second electrode set for separating the opening to a first reaction zone and a second reaction zone, and the first reaction zone is corresponding to the first electrode set and the second reaction zone is corresponding to the second electrode set;

a reaction reagent covered on the first electrode set and corresponding to the first reaction zone that used for reaction with the analyte, wherein the reaction reagent is not covered on the second electrode set and corresponding to the second reaction zone; and

a cover covered on the space.

Preferably, the first electrode set is applied a first signal with a DC signal and the second electrode set is applied a second signal with an AC signal in the biosensor strip in accordance with the present invention. More preferably, the second signal is an AC with DC offset signal and has a constant frequency. Furthermore, the AC signal preferably has amplitude of 0.5 to 2 V and the constant frequency of the AC signal could be less than 5 kHz. Besides, the DC offset is preferably less than 2000 mV and more preferably is 250, 500, 750, 1000, 1500 or 2000 mV. In the preferred embodiment of the present invention, the AC signal is a big signal of 0.5 to 2V and is different from the prior art that utilizes an AC signal of small signal about 12.4 mV to 56.6 mV for testing impedance.

In a preferred embodiment of the present invention, the biosensor strip further may comprise a polymer layer covered on the second reaction zone. More preferably, the polymer layer employed in the present invention is selected from the group consisting of methylcellulose (MC), ethylcellulose (EC), carboxymethyl cellulose (CMC), carboxyethyl cellulose (CEC), methylhydroxyethylcellulose (MHEC), methylhydroxypropylcellulose (MHPC), ethylhydroxyethylcellulose (EHEC), hydroxyethylcellulose (HEC), hydroxypropylcellulose (HPC), hydroxypropylmethylcellulose (HPMC), hydroxyethylcarboxymethylcellulose (HECMC), carboxymethylhydroxyethylcellulose (CMHEC), polyvinylpyrrolidone-10 (PVP-10), polyvinylpyrrolidone-40 (PVP-40), polyvinyl alcohol (PVA), polyamino acid or derivative thereof, polyacrylic acid or salt thereof, starch or derivative thereof, polymethacrylic acid or salt thereof, maleic anhydride polymer or salt thereof, agarose gel or derivation thereof and any one of the combination layer.

In a preferred embodiment of the present invention, the first electrode set of the electrode layer is formed far from an end of the base and the second electrode is formed near the end of the base. More preferably, the electrode layer further comprises a detecting electrode formed far from the end of the base. Furthermore, the electrode layer may comprise a silver layer covered on the base and a carbon layer covered on the silver layer and the first electrode set of the electrode layer may comprise a working electrode and a reference electrode and the second electrode set may comprise a working electrode and a reference electrode. More preferably, the first electrode set and the second electrode set have a common reference electrode.

In another preferred embodiment of the present invention, the electrode layer comprises a first end and a second end, and the first end is near or contacted with a mating biosensing meter and the second end is near or contacted the analyte, and the second end of the carbon layer is not covered on the second end of the silver layer;

Another aspect of the present invention is to provide a biosensing meter with hematocrit correction for inserting a biosensor strip to measure an analyte concentration, the meter comprising:

a connector used for receiving the biosensor strip, wherein the biosensor strip comprises different electrode sets;

a signal applied element used for applying a first signal comprising a DC signal and a second signal comprising an AC signal with a constant frequency to the different electrode sets respectively simultaneously;

a detecting element for detecting the first signal and the second signal to obtain a first response and a second response; and

a microprocessor for receiving the first response and the second response to calculate an uncorrected analyte concentration and hematocrit level respectively and then calculating to obtain a corrected analyte concentration.

Preferably, the biosensing meter employed in the present invention further comprises a memory for storing the uncorrected analyte concentration and the hematocrit level. More preferably, the memory further may comprise a comparison table that compares the first response to uncorrected analyte concentration and the second response to hematocrit level, and the microprocessor calculated the uncorrected analyte concentration and the hematocrit level to obtain the corrected analyte concentration.

In a preferred embodiment of the present invention, the memory of the biosensing meter may comprise three comparison tables, wherein a first comparison table compares a first response to uncorrected analyte concentration, a second table compares a second response to hematocrit level, and a third table compares the uncorrected analyte concentration and the hematocrit level to a corrected analyte concentration. Further, the signal applied element in accordance with the present invention may comprise a DC applied element and an AC applied element. The detecting element employed in the present invention may be a current detecting element.

In another preferred embodiment of the present invention, a waveform of the second signal is square, triangle, trapezoidal or sinusoidal.

In yet a preferred embodiment of the present invention, an analyte concentration measurement system with hematocrit correction is disclosed, the system comprises:

a biosensor strip comprising a electrode layer that comprises a first electrode set and a second electrode set, the first electrode is used for measuring analyte concentration and is covered with a reaction reagent specifically reacted with the analyte and the second electrode set is used for measuring hematocrit level;

a biosensing meter used for selected received the biosensor strip and comprising:
a signal applied element used for applying a first signal comprising a DC signal to the first electrode set and a second signal comprising an AC signal to the second electrode set respectively simultaneously;
a detecting element for detecting the first signal and the second signal to obtain a first response and a second response; and
a microprocessor for receiving the first response and the second response to calculate results respectively and then calculating to obtain the analyte concentration.

The biosensing meter of the system employed in the present invention may further comprise a connector used for receiving the biosensor strip and contacting with the electrode sets.

In another embodiment of the present invention, a measurement method for detecting analyte concentration in a sample with hematocrit correction is disclosed, comprising:

providing a biosensor strip as above mentioned;

providing the sample to contact with the first electrode set and the second electrode set;

applying a first signal to the first electrode set and simultaneously applying a second signal to the second electrode set, wherein the first signal comprises a DC signal and the second signal comprises an AC signal with a constant frequency;

measuring the first signal to obtain a first response;

measuring the second signal to obtain a second response;

calculating the first response to obtain a first measurement value, wherein the first measurement value is uncorrected analyte concentration;

calculating the second response to obtain a second measurement value, wherein the second measurement value is hematocrit level; and

calculating the first measurement value and the second measurement value to obtain a corrected analyte concentration.

Preferably, the constant frequency of the AC signal employed in the method in accordance with the present invention may be less than 5 kHz. More preferably, the constant frequency of the AC signal is 1, 2, 3 or 4 kHz. Further, the AC signal may have amplitude of 0.5 to 2 V. More preferably, the amplitude is 0.5, 1, 1.5 or 2 V.

In another preferred embodiment of the present invention, the AC signal employed in the method may comprise an AC with DC offset voltage and the DC offset is less than 2000 mV. More preferably, the DC offset is 250, 500, 750, 1000, 1500 or 2000 mV.

The AC signal employed in the method of the present invention may have a waveform of square, triangle, trapezoidal or sinusoidal or the AC signal is a continued pulse and the DC signal is set between 300 mV to 600 mV. Further, the first signal and the second signal employed in the present invention may preferably comprise a current information respectively.

In a preferred embodiment of the present invention, the measuring the first signal and measuring the second signal step may be processing simultaneously. More preferably, measuring the first signal and the second signal step is measured during 5 seconds. Furthermore, the method preferably disclosed that

calculating the first response to obtain the first measurement value is comparing the first response to a comparison table to find the corresponded uncorrected analyte concentration;

calculating the second response to obtain the second measurement value is comparing the second response to another comparison table to find the corresponded hematocrit level; and

calculating the first measurement value and the second measurement value is comparing the uncorrected analyte concentration and hematocrit level to another comparison table to find the corresponded corrected analyte concentration.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will be further described, by way of example only, with reference to the accompanying drawings, in which:

FIG. 1 is a diagram of an excitation signal suitable for use in a system and method in accordance with the present invention, having a simultaneously-applied AC signal and DC signal;

FIG. 2 is a perspective view of a first embodiment of a biosensor strip in accordance with the present invention;

FIG. 3 is an exploded perspective view of the biosensor strip in FIG. 2;

FIG. 4 is a diagram of an end of an electrode layer of the biosensor strip in FIG. 2;

FIG. 5 is a diagram of a reaction zone of the biosensor strip in FIG. 2;

FIG. 6 is an exploded perspective view of a second embodiment of a biosensor strip in accordance with the present invention;

FIG. 7 is a plot of a correlation between current versus hematocrit level measured by whole blood and plasma respectively using the biosensor strip with different frequencies and amplitudes for the test of example 1;

FIG. 8 is a plot of a correlation between current versus hematocrit level measured by whole blood and plasma respectively using the biosensor strip with 2 kHz and 4 kHz of frequencies for the test of example 1;

FIG. 9 is a plot of a correlation between current versus hematocrit level for the test of example 2;

FIG. 10 is a plot of a correlation between current versus hematocrit for the test of example 3 by detecting with a reaction reagent covered on a first electrode set and a second electrode set;

FIG. 11 is a plot of a correlation between current versus hematocrit for the test of example 4 by detecting with a reaction reagent covered only one electrode set;

FIG. 12 is a plot of blood glucose concentration detected by the biosensor strip of FIG. 2 versus YSI for the test of example 5; and

FIG. 13 is a plot of a correlation between current versus hematocrit for the test of example 7.

### DESCRIPTION OF THE PREFERRED EMBODIMENTS

For the purposes of promoting an understanding of the principles of the invention, reference will now be made to the embodiment illustrated in the drawings, and specific language will be used to describe that embodiment. It will nevertheless be understood that no limitation of the scope of the invention is intended. Alterations and modifications in the illustrated device, and further applications of the principles of the invention as illustrated therein, as would normally occur to one skilled in the art to which the invention relates are contemplated, are desired to be protected. In particular, although the invention is discussed in terms of a blood glucose meter, it is contemplated that the invention can be used with devices for measuring other analytes and other sample types. Such alternative embodiments require certain adaptations to the embodiments discussed herein that would be obvious to those skilled in the art.

A system, biosensor strip, biosensing meter and method according to the present invention permit the accurate measurement of an analyte in a sample with interfereant correction. The measurement of the analyte remains accurate despite the presence of interferants, which would otherwise cause error, in particular of blood sample. For example, a blood glucose meter according to the present invention measures the concentration of blood glucose without error that is typically caused by variations in the hematocrit level of the sample. The accurate measurement of blood glucose is invaluable to the long term monitoring blood glucose level and prevention of blindness, loss of circulation, and other complications in diabetics. For providing an accurate measurement, the biosensing meter according to the present invention preferably comprises a voltage providing element for providing a first signal comprising a DC component and a second signal comprising an AC component to a sample. In a preferred embodiment of the present invention, the biosensor strip comprises two reaction zones and each has an electrode set, and the first signal and the second signal provided to different reaction zones respectively. Thus, it can detect uncompensated analyte concentration and hematocrit level by different reaction zones to obtain more accurate measurement by compensating the accurate hematocrit level. Another advantage of the system, biosensor strip, biosensing meter and method of the present invention is making the measurement much more rapidly and more convenient.

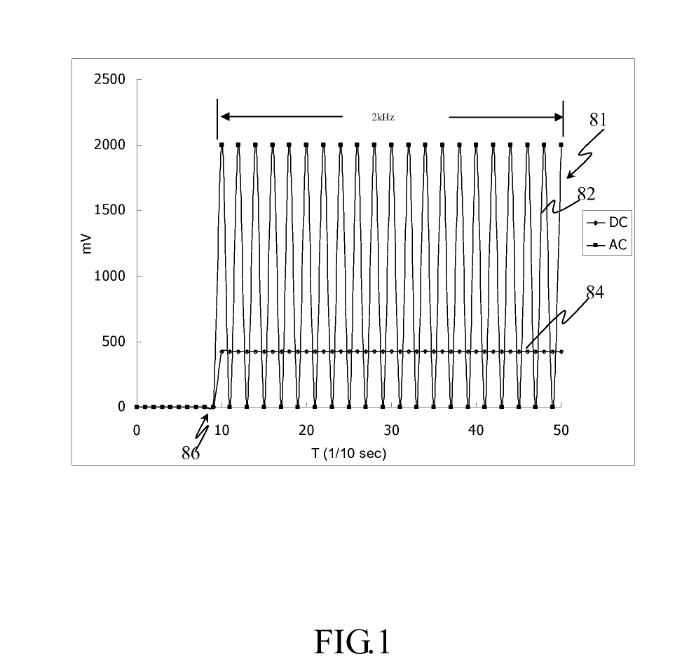

A preferred embodiment of the present invention is provided a method for analyte measurement with interferant correction. The method comprises providing and detecting a response of an AC signal to detect impedance of a sample to calculate a corresponding hematocrit level. Please refer to FIG. 1, which illustrates a preferred embodiment excitation signal suitable for use in a system and method according to the present invention, indicated at 81, in which one AC excitation and DC excitation are used. At time 86, starting providing a first signal (84) that comprises a DC signal and a second signal (82) that comprises an AC signal at the same time. In a preferred embodiment of the present invention, the second signal (82) is an AC with DC offset signal. In the preferred embodiment, the frequency is less than 5 kHz, and has amplitude between 0.5 V to 2 V and an AC with DC offset value is less than 2000 mV. A frequency of 2 kHz and amplitude is 2 V are used in the example of FIG. 1. The AC signal may be used various waveforms, including, for example, square, triangle, trapezoidal, sinusoidal or continued pulse. The DC amplitude is preferably between 300 mV to 600 mV, and it is 425 mV used in the example of FIG. 1.

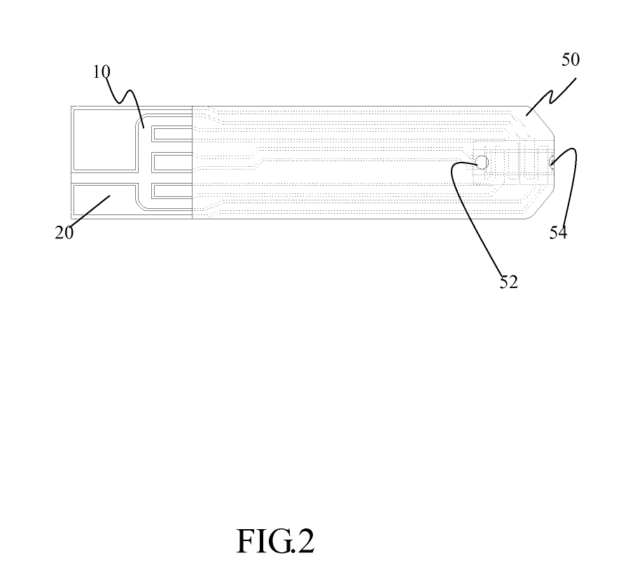

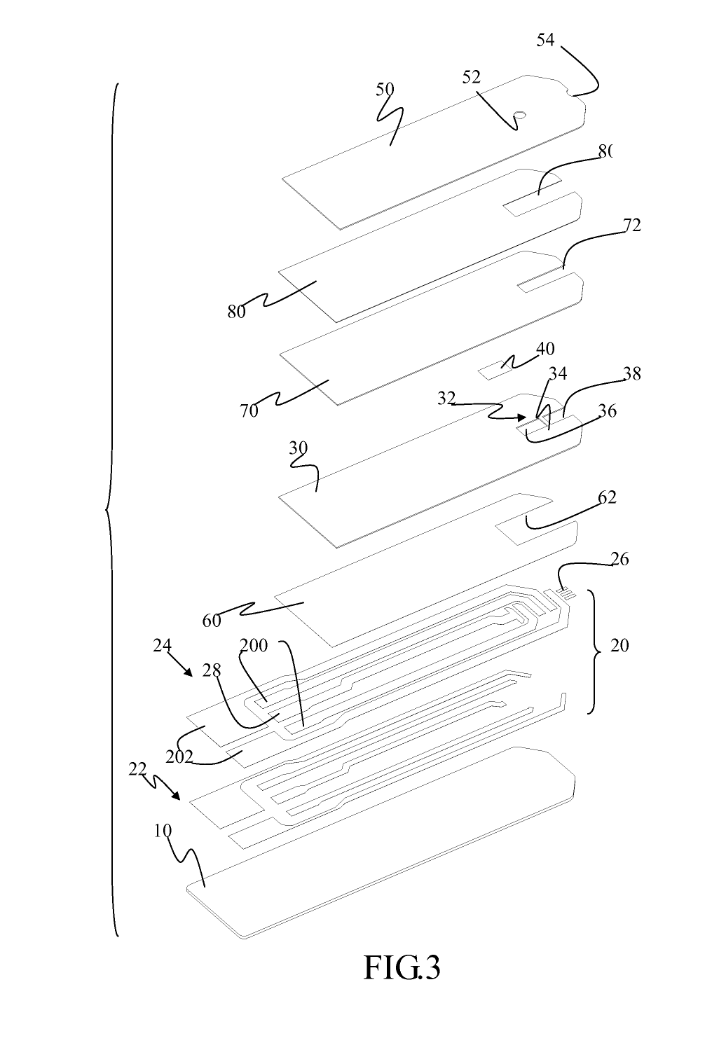

Please referring to FIGS. 2 and 3, it shows a perspective and exploded perspective view of a first embodiment of a biosensor strip. The biosensor strip comprises a base (10), an electrode layer (20), a spacer (30), a reaction reagent (40) and a cover (50).

The base (10) can be preferably an insulating substance and has electrical insulating characteristic.

The electrode layer (20) is laid on the base (10) and comprises a first end, a second end, a first electrode set (200) and a second electrode set (202). The first end of the electrode layer (20) is used for contact with a biosensing meter and the second end of the electrode layer (20) is used for contact a sample.

In a preferred embodiment of the present invention, a method for measurement comprises steps of following:

providing a first signal to the first electrode set (200), which comprises a DC signal; and

providing a second signal to the second electrode set (202), which comprises an AC signal.

The second signal preferably comprises AC signal with DC offset and the waveform of the AC signal could be square, triangle, trapezoidal or sinusoidal. More preferably, the second signal is a continued pulse.

In a preferred embodiment of the present invention, the first electrode set (200) and the second electrode set (202) comprises a working electrode and a reference electrode respectively. In another preferred embodiment of the present invention, the first electrode set (200) and the second electrode set (202) can use the same reference electrode and more preferably, the reference electrode can comprise two ends in contact with the reaction zone and the two ends of the reference electrode are cooperating with the working electrodes of the first electrode set (200) and the second electrode set (202) respectively. Preferably, the electrode layer (20) comprises a silver layer (22) laid on the base (10) and a carbon layer (24) laid on the silver layer (22).

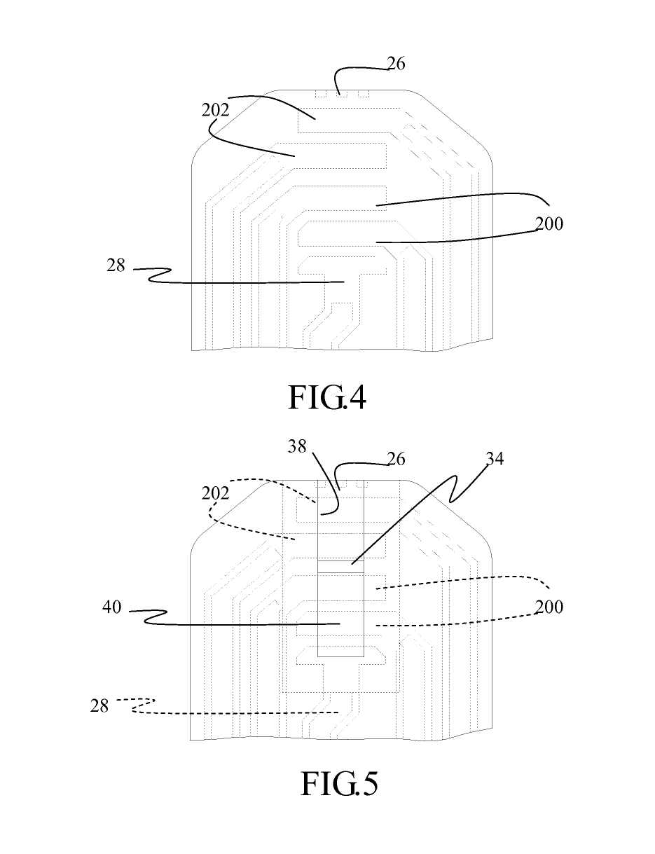

Referring to FIG. 3, the first electrode set (200) can be set on farer from the second end of the electrode layer (20). In a preferred embodiment of the present invention, a detecting electrode (28) is further laid on the base (10) when the first electrode set (200) is set farer from the second end. The detecting electrode (28) is used for detecting a sample and to start the measurement when the sample is contacting with the detecting electrode (28). Further, the detecting electrode (28) can be used for detecting whether the sample volume is enough or not, and therefore, the measurement can start if the sample volume is enough. Further referring to FIG. 4, it shows an enlarged view of one end of the biosensor strip. In a preferred embodiment of the present invention, the second end of the silver layer (22) is not covered on the second end of the carbon layer (24) and therefore, it can prevent silver layer (22) from interfering reaction but can increase transmitting rate at the end far from the reaction reagent (40) to deduce the reaction time.

The spacer (30) is laid on partial base (10) and the electrode layer (20) and exposed an end of the electrode layer (20) for contacting with the biosensor meter. Preferably, the spacer (30) comprises an opening (32) exposed the other end of the electrode layer (20). In a preferred embodiment of the present invention, the opening (32) is set perpendicularly and opened to one end of the spacer (30). In another preferred embodiment of the present invention, the opening (32) can be set horizontally and opened to one side of the spacer (30). More preferably, the spacer (30) further comprises a separated element (34) formed corresponding to the opening (32) for dividing the opening (34) into two zones that are a first reaction zone (36) and a second reaction zone (38). Preferably, the spacer (30) could be formed by printing. The separated element (34) is used for preventing from reaction interfering at the first reaction zone (36) and the second reaction zone (38).

In a preferred embodiment of the present invention, the biosensor strip further comprises an insulation layer (60) set between the spacer (30) and the electrode layer (20). The insulation layer (60) comprises a second opening (62) corresponding to the opening (32) of the spacer (30) for exposing a part of the electrode layer (20).

In another preferred embodiment of the present invention, the biosensor strip further comprises a second insulation layer (70) set on the spacer (30). The second insulation layer (70) comprises a third opening (72) corresponding to the opening (32) of the spacer (30).

Further referring to FIG. 5, the reaction reagent (40) is covered on the first reaction zone (36) of the opening (32) and on an end of the first electrode set (200). The reaction reagent (40) is specific for the test to be performed by the strip and contains biological activated material (ex. Enzyme), enzyme cofactor, stabilizer (ex. macromolecule polymer), buffer and so on. Preferably, the reaction reagent (40) is not covered on the second reaction zone (38).

When the reaction reagent (40) is covered on the first electrode set (200) and not covered on the second electrode set (202), hematocrit measurement could prevent from interfering of the reaction reagent (40) which can be supported by following example. When the sample received in the strip, it will contact with the second electrode set (202) firstly and then pass to the first reaction zone (36) to contact with the first electrode set (200), and finally arrive to the detecting electrode (28). At the time of the detecting electrode (28) detects the sample, the measurement is starting.

In another preferred embodiment of the present invention, it could have other materials on the second electrode (202). Preferably, the biosensor strip further comprises a polymer layer on the second reaction zone (38) and on the exposed second electrode set (202). The polymer layer could be selected from the group consisting of methylcellulose (MC), ethylcellulose (EC), carboxymethyl cellulose (CMC), carboxyethyl cellulose (CEC), methylhydroxyethylcellulose (MHEC), methylhydroxypropylcellulose (MHPC), ethylhydroxyethylcellulose (EHEC), hydroxyethylcellulose (HEC), hydroxypropylcellulose (HPC), hydroxypropylmethylcellulose (HPMC), hydroxyethylcarboxymethylcellulose (HECMC), carboxymethylhydroxyethylcellulose (CMHEC), polyvinylpyrrolidone-10 (PVP-10), polyvinylpyrrolidone-40 (PVP-40), polyvinyl alcohol (PVA), polyamino acid or derivative thereof, polyacrylic acid or salt thereof, starch or derivative thereof, polymethacrylic acid or salt thereof, maleic anhydride polymer or salt thereof, agarose gel or derivation thereof and any one of the combination. The polymer layer is used for increasing the accuracy of hematocrit measurement.

In a preferred embodiment of the present invention, the biosensor strip further comprises a hydrophilic layer set under the reaction reagent (40) corresponding to the opening (32) for increasing the stability of materials on the base (10). The hydrophilic layer is used for increasing attachment effective of the reaction reagent (40).

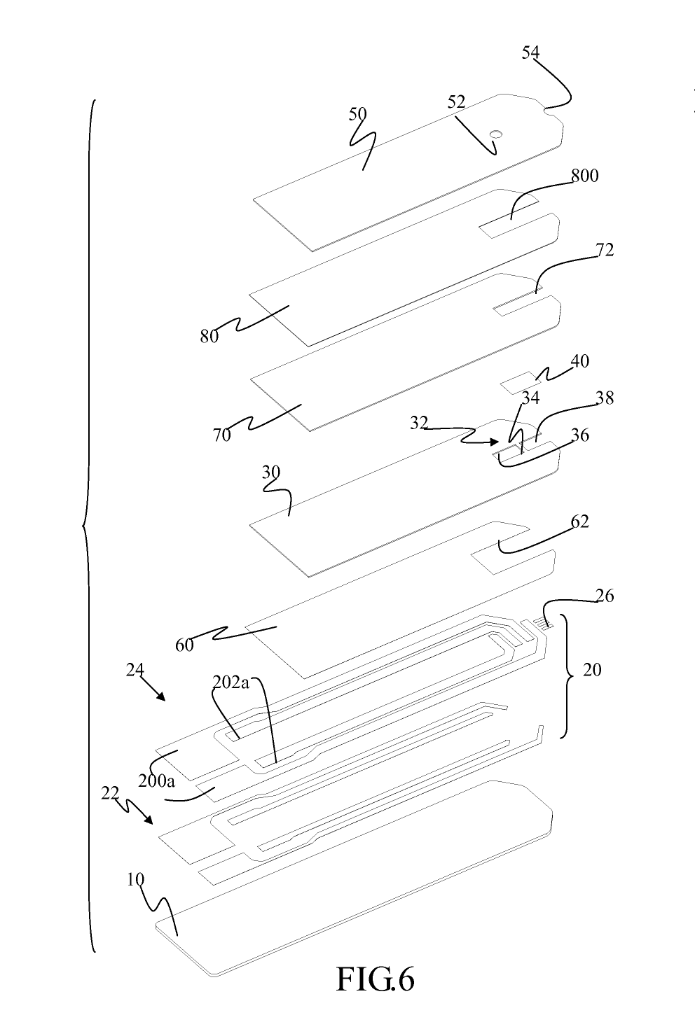

Further refer to FIG. 6, the first electrode set (200a) could set near the second end of the electrode layer (20). More preferably, the first electrode set (200a) set near the second end and the reference electrode of the second electrode set (202a) could also be a detecting electrode for detecting whether a sample volume is enough.

In a preferred embodiment of the present invention, the biosensor strip further has a rough unit (26) laid on the base (10) and located corresponding to the opening (32) of the spacer (30). Preferably, the rough unit (26) is a line or multiple lines and more preferably laid on an outside and adjacent to the second end of the electrode layer (20). The rough unit (26) is preferably prepared by electric conduction substance or non-electric conduction substance. More preferably, the rough unit (26) is prepared by carbon and separated from the electrode system (20). The rough unit (26) can increase the rough of the base (10), and therefore, the reaction reagent (40) laid on the base (10) will not easy to be shaking off when the strip is cut for separating.

The cover (50) is covered on the spacer (30) and has a hole (52) corresponding to the opening (32) of the spacer (30). Preferably, the hole (52) is corresponding far from the second end. Furthermore, the cover (50) further has a concave unit (54) that is formed corresponding to outside of the opening (32) of the spacer (30). Preferably, the biosensor strip further comprises an adhesive layer (80) for adhering the cover (50) to the spacer (30). The adhesive layer (80) comprises a fourth opening (800) corresponding to the opening (32) of the spacer (30).

In a preferred embodiment of the present invention, the biosensing meter comprises a connector, a signal applied element, a detecting element and a microprocessor. The biosensor strip firstly inserts into the connector of the biosensing meter when measurement. Please referring to FIG. 1, the signal applied element provides a voltage and the biosensing meter detects whether a sample volume is enough or not. Turn off the power in a period of time and then the biosensing meter provides a first signal to the first electrode set and a second signal to the second electrode set at the same time. Preferably, the first signal is a DC signal and the second signal is an AC with DC offset signal. The DC offset is less then 2000 mV and more preferably, the DC offset is 2000, 1500, 1000, 750, 500, 250 mV. Detect a response of the first signal from the first electrode set and a response of the second signal from the second electrode set by the detecting element. In a preferred embodiment of the present invention, the detecting element is a current detecting element and measuring a current. Due to the current and impedance has a relationship and it is a positive correlation between the hematocrit level and impedance, measuring impedance could calculate a corresponding hematocrit level. Further, the impedance is a ratio of voltage and current, and therefore, hematocrit level could calculate from a reciprocal of the current when providing the same voltage.

It will be appreciated that the response may be measured as current or voltage and the impedance can be calculated therefrom. Although the present specification and claims may refer alternately to the AC response as impedance, resistance, conductivity, current or charge, and to the DC response as current, charge, resistance or conductivity, those skilled in the art will recognize that these measures are interchangeable, it only being necessary to adjust the measurement and correction mathematics to account for which measure is being employed.

A preferred embodiment of the present invention provides a system for preventing from hematocrit interference. The system comprises above mentioned biosensing meter and biosensor strip and the analyte concentration is measured accurately by means of the cooperation between the biosensing meter and biosensor strip.

The system and method in accordance with the present invention has following advantages.

1. The biosensor strip according to the present invention comprises two different electrode sets for measurement of analyte concentration and hematocrit level respectively, and therefore, the response of the above two will not interfere from each other.

2. The biosensor strip according to the present invention comprises two reaction zones for measurement of analyte concentration and hematocrit level respectively and thus it can completely separate the two reactions for increasing measurement accuracy.

3. The biosensor strip according to the present invention comprises different layers on the two reaction zones, for example, a reaction reagent on analyte concentration reaction zone and polymer layer on hematocrit reaction zone, and therefore, it can specifically increase hematocrit measurement accuracy.

4. The biosensing meter according to the present invention provides the first signal and the second signal simultaneously to the two different electrode sets respectively for achieving analyte concentration and hematocrit level measurement separately but also achieving rapid reaction time and accurate measurement.

5. The biosensing meter according to the present invention provides the AC signal with a set specifically constant frequency for achieving rapid reaction time and simple design, and decreasing energy consumption. Further, the AC signal with a big amplitude for testing could obtain a linear like relationship between current and hematocrit level.

It will be appreciated that a method according to the present invention may also be used to measure the concentration of other analytes and in other fluids. For example, a method according to the present invention may be used to measure the concentration of a medically significant analyte in urine, saliva, spinal fluid, etc. Likewise, by appropriate selection of reagent a method according to the present invention may be adapted to measure the concentration of, for example, lactic acid, uric acid, etc.

Although the following examples deal with correcting for the interfering effects of hematocrit level on blood glucose determinations, those skilled in the art will recognize that the teaching of the present invention is equally useful for correcting for the effects of other interferants in both blood glucose measurement and in the measurement of other analytes. Furthermore, the present specification and claims refer to steps such as “determine/detect the hematocrit level”. To use the hematocrit level as an example, it is intended that such statement includes not only determining/detect the actual hematocrit level, but also a hematocrit correction factor vs. some nominal point. In other words, the process may never actually arrive at a number equal to the hematocrit level of the sample, but instead determine that the sample's hematocrit differs from a nominal value by a certain amount. Both concepts are intended to be covered by statements such as “determine/detect the hematocrit level.”

The following examples are illustrative of the principles and practice of this invention. Numerous additional embodiments within the scope and spirit of the invention will become apparent to those skilled in the art.

### Example 1

### Whole Blood and Plasma Measurement with Different Frequencies, Amplitudes and Time of the Excitation to Obtain Hematocrit Level

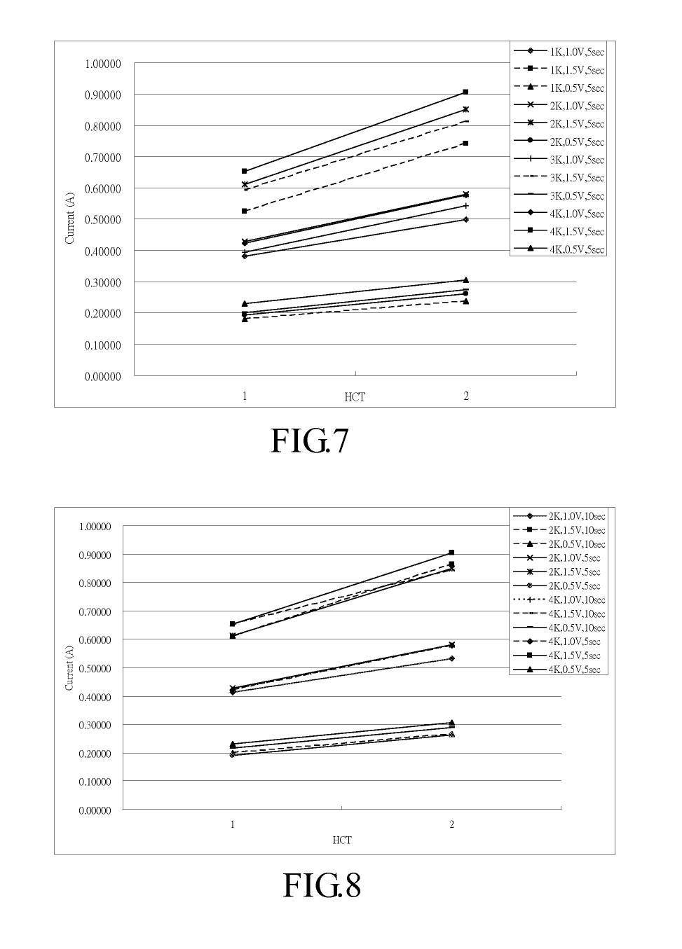

This example applied an AC with DC offset signal with different frequencies and different amplitudes to a sample within a biosensor strip. The frequencies were 1, 2, 3 and 4 kHz and the amplitudes were 0.5, 1 and 1.5V. The samples were whole blood and plasma and detect the current values at 5 and 10 seconds. The hematocrit levels were calculated and the result was shown in FIGS. 7 and 8.

Referring to FIG. 7, left is whole blood result and right is plasma result. Detect the current values after 5 seconds when apply an AC signal with different frequencies and amplitudes. Compare difference between whole blood and plasma.

Referring to FIG. 8, left is whole blood result and right is plasma result. Detect the current values after 5 and 10 seconds when apply an AC signal with 2 and 4 kHz frequencies and different amplitudes. Compare the difference between whole blood and plasma.

### Example 2

### Current Versus Hematocrit Relationship Measurement

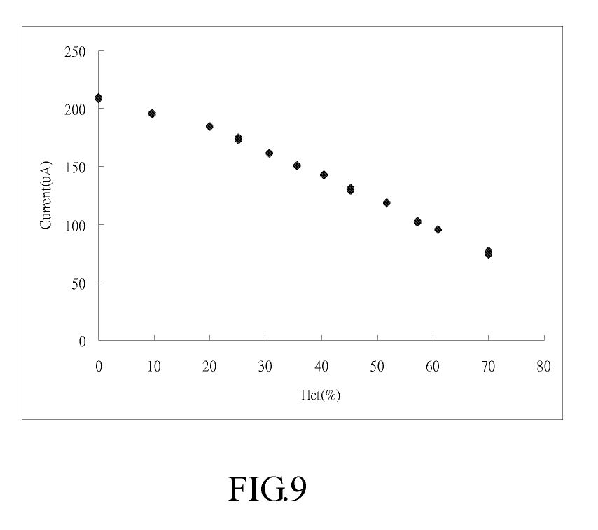

This example used biosensor strips like FIG. 2 of the present invention. Apply an AC with DC offset signal to samples within biosensor strips. The signal comprised a frequency of 2 kHz and amplitude of 1 V. Detect the current value after 5 seconds with different hematocrit levels of samples and repeat three times. Compare the current and the hematocrit levels and the result was shown in FIG. 9.

Referring to FIG. 9, it can obtain a relationship curve between current and hematocrit level by measuring the sample three times and measuring different samples.

### Example 3

### Measurement Hematocrit Level by Utilizing a Reaction Reagent Covered on a First Electrode Set and a Second Electrode Set of a Biosensor Strip

This example utilized a biosensor strip like FIG. 2 except a reaction reagent is covered on the first electrode set and the second electrode set. The hematocrit levels of samples are 0, 20, 45 and 65%.

When measurement, sample received by capillarity to the reaction reagent of the biosensor strip. Apply a DC signal to the first electrode set and an AC with DC offset signal to the second electrode set, and then measure the current value at the desired time to repeat eight times.

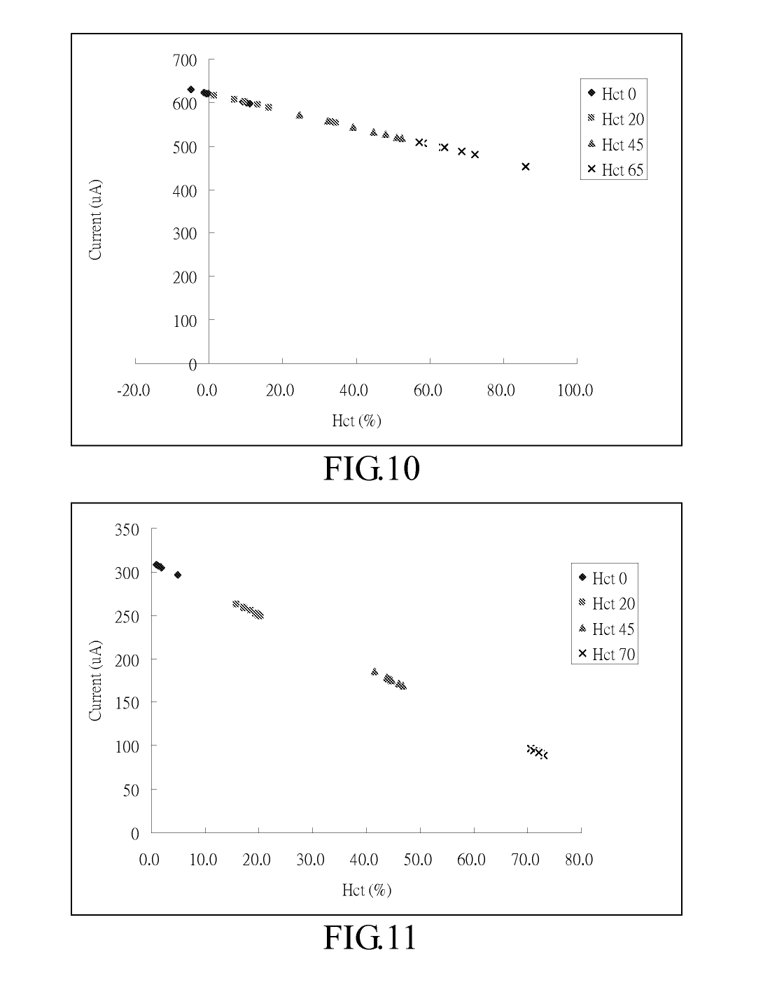

Referring to FIG. 10, it is a current and hematocrit relationship curve of this example. The result showed a big deviation if the reaction reagent is covered on the first electrode set and the second electrode set.

### Example 4

### Measurement Hematocrit Level by Utilizing a Biosensor Strip that has no Reaction Reagent on the Electrode Set for Detecting the Hematocrit Level

This example utilized a biosensor strip like FIG. 2. The hematocrit levels of samples are 0, 20, 45 and 70%.

When measurement, sample received by capillarity to the reaction reagent of the biosensor strip. Apply a DC signal to the first electrode set and an AC with DC offset signal to the second electrode set, and then measure the current value at the desired time repeated eight times.

Referring to FIG. 11, it is a current and hematocrit relationship curve of this example. The result showed a narrow deviation than that of example 3. Thus, it can obtain accurate hematocrit levels if the reaction reagent is not covered on the second electrode set for preventing from reaction reagent interfering.

### Example 5

### Measurement Blood Glucose Value by Utilizing a Biosensor Strip that has no Reaction Reagent on the Electrode Set for Detecting Hematocrit Level

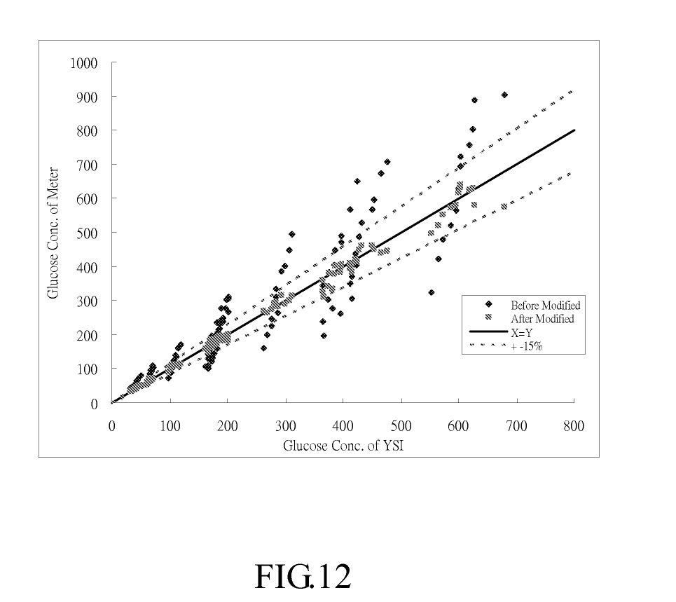

The example used biosensor strips like FIG. 2 of the present invention. The biosensor strip had reaction reagent on the first electrode set for detecting blood glucose concentration and not on the second electrode set for detecting hematocrit level. Provide 120 samples for testing and compare to YSI measurement. Referring to FIG. 12, further compare blood glucose concentration with hematocrit corrected and uncorrected. The result showed that hematocrit corrected blood glucose concentration is more accurate.

### Example 6

### Simultaneously Applied AC with DC Offset and DC Signal and in any Order of AC with DC Offset then DC or DC then AC with DC Offset

This example used biosensor strip like FIG. 2 of the present invention for three different tests. Firstly, simultaneously applied an AC with DC offset signal and a DC signal to different electrode sets respectively. The AC with DC offset signal is applied to the second electrode set without reaction reagent covered for hematocrit level measurement and the DC signal is applied to the first electrode set with reaction reagent for blood glucose measurement. Secondly, applied the AC with DC offset signal to the second electrode set and then applied the DC signal to the first electrode set. And thirdly, applied the DC signal to the first electrode set and then applied the AC with DC offset signal to the second electrode set. After calculating, compare blood glucose concentration with YSI result. Following tables show the CV values measured by the above three methods.

TABLE 1
AC and DC simultaneously

| HCT (%) | | |
|---|---|---|
| 0 | CV | 1.8% |
| | Diff. of YSI | 2.6% |
| 14~20 | CV | 2.2% |
| | Diff. of YSI | −3.4% |
| 44~45 | CV | 2.0% |
| | Diff. of YSI | 0.0% |
| 60~65 | CV | 3.4% |
| | Diff. of YSI | −2.8% |

TABLE 2
AC and then DC

| HCT (%) | | |
|---|---|---|
| 0 | CV | 6.6% |
| | Diff. of YSI | 8.4% |
| 14~20 | CV | 21.6% |
| | Diff. of YSI | −2.6% |
| 44~45 | CV | 19.1% |
| | Diff. of YSI | 0.0% |
| 60~65 | CV | 9.7% |
| | Diff. of YSI | −20.2% |

TABLE 3
DC and then AC

| HCT (%) | | |
|---|---|---|
| 0 | CV | 3.3% |
| | Diff. of YSI | 57.2% |
| 14~20 | CV | 9.1% |
| | Diff. of YSI | 23.4% |
| 44~45 | CV | 11.5% |
| | Diff. of YSI | 0.0% |
| 60~65 | CV | 8.2% |
| | Diff. of YSI | −8.0% |

The above three tables show that simultaneously applied two signals is more accurate than applied AC with DC offset signal and DC signal in any order.

### Example 7

### Correlation of Hematocrit Level and Measured Current Value by Different Blood Glucose Concentration Sample

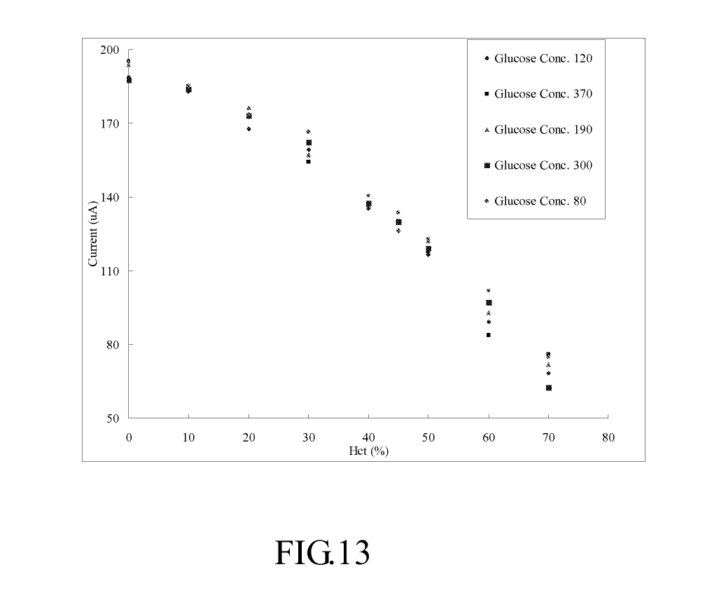

This example used five different blood glucose concentration samples and the concentrations are 80, 120, 190, 300 and 370 mg/dl. Every concentration has nine hematocrit levels for testing correlation between hematocrit level and current. The nine hematocrit levels are 0, 10, 20, 30, 45, 50, 60 and 70%. This example used biosensor strips like FIG. 2 of the present invention. Apply a big signal of AC excitation to the second electrode set for testing hematocrit level versus current and the result showed in FIG. 13. Because of the unceasing experimental accumulation, then obtains relational graph of the magnitude of current and the hematocrit level according to the empirical rule and shows about a linear relationship.

Other embodiments of the invention will appear to those skilled in the art from consideration of the specification and practice of the invention disclosed herein. It is intended that the specification and examples to be considered as exemplary only, with a true scope and spirit of the invention being indicated by the following claims.

What is claimed is:

1. A biosensor strip for measurement an analyte concentration, comprising:
a base;
an electrode layer covered on the base and comprising a first electrode set and a second electrode set, wherein the first electrode set is used for measurement analyte concentration and the second electrode set is used for measurement hematocrit level;
a space covered on the electrode layer and exposed an end of the electrode layer and comprising an opening, wherein the opening exposed another end of the electrode layer, and further comprises a separated element formed corresponding to and in the middle of the first electrode set and the second electrode set for separating the opening to a first reaction zone and a second reaction zone, and the first reaction zone is corresponding to the first electrode set and the second reaction zone is corresponding to the second electrode set;
a reaction reagent covered on the first electrode set and corresponding to the first reaction zone that used for reaction with the analyte, wherein the reaction reagent is not covered on the second electrode set and corresponding to the second reaction zone; and
a cover covered on the space.

2. The biosensor strip as claimed in claim 1, wherein the first electrode set is applied a first signal with a DC signal and the second electrode set is applied a second signal with an AC signal.

3. The biosensor strip as claimed in claim 2, wherein the second signal is an AC with DC offset signal and has a constant frequency.

4. The biosensor strip as claimed in claim 3, wherein the AC signal has amplitude of 0.5 to 2 V.

5. The biosensor strip as claimed in claim 4, wherein the constant frequency of the AC signal is less than 5 kHz.

6. The biosensor strip as claimed in claim 5, wherein the DC offset is less than 2000 mV.

7. The biosensor strip as claimed in claim 6, wherein the DC offset is 250, 500, 750, 1000, 1500 or 2000 mV.

8. The biosensor strip as claimed in claim 7, further comprising a polymer layer covered on the second reaction zone.

9. The biosensor strip as claimed in claim 8, wherein the polymer layer is selected from the group consisting of methylcellulose (MC), ethylcellulose (EC), carboxymethyl cellulose (CMC), carboxyethyl cellulose (CEC), methylhydroxyethylcellulose (MHEC), methylhydroxypropylcellulose (MHPC), ethylhydroxyethylcellulose (EHEC), hydroxyethylcellulose (HEC), hydroxypropylcellulose (HPC), hydroxypropylmethylcellulose (HPMC), hydroxyethylcarboxymethylcellulose (HECMC), carboxymethylhydroxyethylcellulose (CMHEC), polyvinylpyrrolidone-10 (PVP-10), polyvinylpyrrolidone-40 (PVP-40), polyvinyl alcohol (PVA), polyamino acid or derivative thereof, polyacrylic acid or salt thereof, starch or derivative thereof, polymethacrylic acid or salt thereof, maleic anhydride polymer or salt thereof, agarose gel or derivation thereof and any one of the combination layer.

10. The biosensor strip as claimed in claim 9, wherein the first electrode set of the electrode layer is formed far from an end of the base and the second electrode is formed near the end of the base and the electrode layer further comprises a detecting electrode formed far from the end of the base.

11. The biosensor strip as claimed in claim 1, wherein the electrode layer comprises a silver layer covered on the base and a carbon layer covered on the silver layer and wherein the first electrode set of the electrode layer comprises a working electrode and a reference electrode and the second electrode set comprises a working electrode and a reference electrode.

12. The biosensor strip as claimed in claim 11, wherein the first electrode set and the second electrode set have a common reference electrode.

13. The biosensor strip as claimed in claim 12, wherein the electrode layer comprises a first end and a second end, and the first end is near or contacted with a mating biosensing meter and the second end is near or contacted the analyte, and the second end of the carbon layer is not covered on the second end of the silver layer;

14. A biosensing meter with hematocrit correction for inserting a biosensor strip to measure an analyte concentration, comprising:
a connector used for receiving the biosensor strip, wherein the biosensor strip comprises different electrode sets;
a signal applied element used for applying a first signal comprising a DC signal and a second signal comprising an AC signal with a constant frequency to the different electrode sets respectively simultaneously;
a detecting element for detecting the first signal and the second signal to obtain a first response and a second response; and
a microprocessor for receiving the first response and the second response to calculate an uncorrected analyte concentration and hematocrit level respectively and then calculating to obtain a corrected analyte concentration.

15. The biosensing meter as claimed in claim 14, wherein the second signal comprises an AC with DC offset signal or is a continued pulse.

16. The biosensing meter as claimed in claim 15, wherein the AC signal has amplitude of 0.5 to 2 V.

17. The biosensing meter as claimed in claim 16, wherein the AC signal has a constant frequency that is less than 5 kHz.

18. The biosensing meter as claimed in claim 17, wherein the DC offset is less than 2000 mV.

19. The biosensing meter as claimed in claim 18, wherein the DC offset is 250, 500, 750, 1000, 1500 or 2000 mV.

20. The biosensing meter as claimed in claim 14, further comprising a memory for storing the uncorrected analyte concentration and the hematocrit level.

21. The biosensing meter as claimed in claim 20, wherein the memory further comprises a comparison table that compares the first response to uncorrected analyte concentration and the second response to hematocrit level, and the microprocessor calculated the uncorrected analyte concentration and the hematocrit level to obtain the corrected analyte concentration.

22. The biosensing meter as claimed in claim 21, wherein the memory comprises three comparison tables, wherein a first comparison table compares a first response to uncorrected analyte concentration, a second table compares a second response to hematocrit level, and a third table compares the uncorrected analyte concentration and the hematocrit level to a corrected analyte concentration.

23. The biosensing meter as claimed in claim 22, wherein a waveform of the second signal is square, triangle, trapezoidal or sinusoidal, and the signal applied element comprises a DC applied element and an AC applied element, and the detecting element is a current detecting element.

24. An analyte concentration measurement system with hematocrit correction, comprising:
a biosensor strip comprising a electrode layer that comprises a first electrode set and a second electrode set, the first electrode is used for measuring analyte concentration and is covered with a reaction reagent specifically reacted with the analyte and the second electrode set is used for measuring hematocrit level;
a biosensing meter used for selected received the biosensor strip and comprising:
a signal applied element used for applying a first signal comprising a DC signal to the first electrode set and a second signal comprising an AC signal to the second electrode set respectively simultaneously;
a detecting element for detecting the first signal and the second signal to obtain a first response and a second response; and
a microprocessor for receiving the first response and the second response to calculate results respectively and then calculating to obtain the analyte concentration.

25. The system as claimed in claim 24, wherein the biosensing meter further comprises a connector used for receiving the biosensor strip and contacting with the electrode sets.

26. The system as claimed in claim 25, wherein the biosensor strip further comprises a spacer covered on the electrode layer and exposed an end of the electrode layer and comprising an opening;
wherein the opening exposed another end of the electrode layer and further comprises a separated element formed corresponding to and in the middle of the first electrode set and the second electrode set for separating the opening to a first reaction zone corresponding to the first electrode set and a second reaction zone corresponding to the second electrode set.

27. The system as claimed in claim 26, wherein the biosensor strip further comprises a polymer layer covered on the second electrode set corresponding to the second reaction zone.

28. The system as claimed in claim 26, wherein the second signal comprises an AC with DC offset.

29. The system as claimed in claim 26, wherein the biosensing meter further comprises a memory that comprises a comparison table that compares the first response to uncorrected analyte concentration and the second response to hematocrit level, and the microprocessor calculates the uncorrected analyte concentration and hematocrit level to obtain a corrected analyte concentration.

30. The system as claimed in claim 29, wherein the memory comprises three comparison tables, and wherein a first comparison table compares a first response to uncorrected analyte concentration, a second comparison table compares a second response to hematocrit level, and a third comparison table compares the uncorrected analyte concentration and hematocrit level to a corrected analyte concentration.

31. A measurement method for detecting analyte concentration in a sample with hematocrit correction, comprising:
providing a biosensor strip as claimed in claim 1;
providing the sample to contact with the first electrode set and the second electrode set;
applying a first signal to the first electrode set and simultaneously applying a second signal to the second electrode set, wherein the first signal comprises a DC signal and the second signal comprises an AC signal with a constant frequency;
measuring the first signal to obtain a first response;
measuring the second signal to obtain a second response;
calculating the first response to obtain a first measurement value, wherein the first measurement value is uncorrected analyte concentration;
calculating the second response to obtain a second measurement value, wherein the second measurement value is hematocrit level; and
calculating the first measurement value and the second measurement value to obtain a corrected analyte concentration.

32. The method as claimed in claim 31, wherein the constant frequency of the AC signal is less than 5 kHz and the AC signal has amplitude of 0.5 to 2 V.

33. The method as claimed in claim 32, wherein the amplitude is 0.5, 1, 1.5 or 2 V.

34. The method as claimed in claim 31, wherein the AC signal comprises an AC with DC offset voltage and the DC offset is less than 2000 mV.

35. The method as claimed in claim 34, wherein the DC offset is 250, 500, 750, 1000, 1500 or 2000 mV and the DC signal is set between 300 mV to 600 mV.

36. The method as claimed in claim 35, wherein the AC signal has a waveform of square, triangle, trapezoidal or sinusoidal or the AC signal is a continued pulse.

37. The method as claimed in claim 31, wherein the first signal and the second signal comprises a current information respectively and measuring the first signal and measuring the second signal is processing simultaneously.

38. The method as claimed in claim 37, wherein measuring the first signal and the second signal during 5 seconds.

39. The method as claimed in claim 31, wherein
calculating the first response to obtain the first measurement value that is comparing the first response to a comparison table to find the corresponded uncorrected analyte concentration;
calculating the second response to obtain the second measurement value that is comparing the second response to another comparison table to find the corresponded hematocrit level; and
calculating the first measurement value and the second measurement value that is comparing the uncorrected analyte concentration and hematocrit level to another comparison table to find the corresponded corrected analyte concentration.
